SEQUENCE LISTING (15 sequences)

SEQ ID NO: 1 - DNA, 615 bp, Homo sapiens
  source 1..615: mol_type = genomic DNA
ttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcagcgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtc

SEQ ID NO: 2 - DNA, 23 bp, synthetic construct
  source 1..23: mol_type = other DNA
ccgtgccgggcgggagaccgcca

SEQ ID NO: 3 - DNA, 21 bp, synthetic construct
  source 1..21: mol_type = other DNA
tgccgggacgggtccaagatg

SEQ ID NO: 4 - DNA, 22 bp, synthetic construct
  source 1..22: mol_type = other DNA
tgggttgctgggtcactctgtc

SEQ ID NO: 5 - DNA, 21 bp, synthetic construct
  source 1..21: mol_type = other DNA
cagcacagaactgcagacagc

SEQ ID NO: 6 - DNA, 21 bp, synthetic construct
  source 1..21: mol_type = other DNA
agacccaagacacaggacagc

SEQ ID NO: 7 - DNA, 19 bp, synthetic construct
  source 1..19: mol_type = other DNA
tagacgaggttggggctgc

SEQ ID NO: 8 - DNA, 19 bp, synthetic construct
  source 1..19: mol_type = other DNA
acgactcgttcacgcctgc

SEQ ID NO: 9 - DNA, 18 bp, synthetic construct
  source 1..18: mol_type = other DNA
ccaatgtctgtccacagc

SEQ ID NO: 10 - DNA, 18 bp, synthetic construct
  source 1..18: mol_type = other DNA
ttcacgccattctcctgc

SEQ ID NO: 11 - DNA, 20 bp, synthetic construct
  source 1..20: mol_type = other DNA
tgggagtctcactctgttgc

SEQ ID NO: 12 - DNA, 20 bp, synthetic construct
  source 1..20: mol_type = other DNA
ctgggtttcaccgtgttagc

SEQ ID NO: 13 - DNA, 53 bp, synthetic construct
  source 1..53: mol_type = other DNA
ggcctccggggactgccgtgccgggcgggagaccgccatggcgaccctggaaa

SEQ ID NO: 14 - DNA, 53 bp, synthetic construct
  source 1..53: mol_type = other DNA
ccggaggcccctgacggcacggcccgccctctggcggtaccgctgggaccttt

SEQ ID NO: 15 - DNA, 23 bp, synthetic construct
  source 1..23: mol_type = other DNA
tggcggtctcccgcccggcacgg

(19) United States
(12) Patent Application Publication
(10) Pub. No.: US 2026/0115318 A1
(43) Pub. Date: Apr. 30, 2026

(54) GENE CORRECTION INDUCED NEURAL STEM CELLS USING HUNTINGTIN GENE-SPECIFIC GUIDE RNA, AND CELL THERAPEUTIC AGENT USING THE SAME

(71) Applicant: KOREA UNIVERSITY RESEARCH AND BUSINESS FOUNDATION, Seoul (KR)

(72) Inventors: Sunghoi HONG, Seoul (KR); Yong NAM-KUNG, Seoul (KR); Donghun HYUN, Seoul (KR)

(73) Assignee: KOREA UNIVERSITY RESEARCH AND BUSINESS FOUNDATION, Seoul (KR)

(21) Appl. No.: 19/362,743

(22) Filed: Oct. 20, 2025

Related U.S. Application Data
(63) Continuation-in-part of application No. PCT/KR2024/005314, filed on Apr. 19, 2024.

(30) Foreign Application Priority Data
Apr. 19, 2023 (KR) 10-2023-0051486

(51) Int. Cl. A61K 48/00; A61K 35/30; A61P 25/28; C12N 5/00; C12N 5/0797; C12N 9/22; C12N 15/11

(52) U.S. Cl. CPC A61K 48/005; A61K 35/30; A61P 25/28; C12N 5/0018; C12N 5/0623; C12N 9/226; C12N 15/111; C12N 2310/20; C12N 2501/148; C12N 2501/16; C12N 2501/41; C12N 2501/727; C12N 2501/73; C12N 2501/845; C12N 2506/1307; C12N 2510/00

(57) ABSTRACT

The present invention relates to: a gene editing composition comprising a guide RNA for editing mutated CAG repeat sequences of neural stem cells (ciNSCs) induced from somatic cells of a Huntington's disease patient; the edited ciNSCs; and a cell therapeutic agent for Huntington's disease, comprising same, wherein, in the edited ciNSCs, Huntington gene mutations are removed by the guide RNA of the present invention, and thus treatment for Huntington's disease and neuronal proliferation can be improved.

### CROSS-REFERENCE TO RELATED APPLICATIONS

This application is a Bypass Continuation-in-Part of International Application No. PCT/KR2024/005314 filed Apr. 19, 2024, which claims priority to Korean Patent Application No. 10-2023-0051486 filed Apr. 19, 2023, the entire disclosures of which are incorporated herein by reference.

### GOVERNMENT SUPPORT STATEMENT

This invention was supported by the Ministry of Science and ICT (MSIT) of the Republic of Korea through the National Research Foundation of Korea (NRF) under Grant Nos. 2019M3E5D5065399 and 2020R1A2C1101294, as part of the Bio & Medical Technology Development Program (“Development of safe and effective patient cell-derived induced neural stem cells for practical application”) and the Basic Research Program (“Investigation of pathogenic mechanisms and discovery of therapeutic factors in Huntington's disease”), respectively, conducted at Korea University. The Government of the Republic of Korea has no rights in this invention.

### INCORPORATION BY REFERENCE OF SEQUENCE LISTING

The content of the electronically submitted sequence listing, file name: Q314683_substitute sequence_listing_as_filed; size: 14,655 bytes; and date of creation: Oct. 30, 2025, filed herewith, is incorporated herein by reference in its entirety.

### TECHNICAL FIELD

The present invention relates to a gene correction composition including a guide RNA for correcting a mutated CAG repeat sequence in neural stem cells (ciNSCs) induced from somatic cells of a Huntington's disease patient; the corrected induced neural stem cells (ciNSCs); and a cell therapeutic agent for Huntington's disease including the same.

### BACKGROUND ART

Huntington's disease is, as a genetic disorder, a genetic neurodegenerative disorder caused by an excessive increase in CAG repeats due to a mutation in the Huntington (HTT) gene located on chromosome 4. Common symptoms of Huntington's disease, which usually develops during adulthood, include involuntary movements, abnormal gait, slurred speech, dysphagia which is difficulty in swallowing food properly, cognitive impairment, personality disorders, and the like.

Gene correction technology has been studied since before CRISPR Cas9. The first-generation gene correction technology is zinc finger technology, which includes a DNA binding domain and a DNA cleavage domain that are structurally stabilized by zinc ions, and recognizes specific base sequences in a protein and cleaves the specific base sequences at specific positions.

However, the zinc finger technology has the disadvantages of low accuracy and high costs. The second-generation gene correction technology is TALEN, which is technology developed by replacing the DNA binding domain of zinc finger technology with TALE protein instead of ZFN. The protein was discovered in the plant pathogenic bacterium Xanthomonas, and is more sophisticated than the first-generation technology, as one protein recognizes one base, but the protein has the disadvantages in that it is difficult to be injected into cells due to its large size, the binding site needs to start with a T base, and it cannot function on methylated C. CRISPR-Cas9 technology, which is third-generation gene correction technology, is a DNA sequence found in the genes of prokaryotic organisms such as bacteria and archaea, and Cas9 is an enzyme that recognizes and cleaves specific strands of DNA complementary to the CRISPR sequence. CRISPR Cas9 includes an RNA-based guide RNA and Cas9, an enzyme that delivers DNA, and can be applied to all cells. CRISPR Cas9 technology is a tool in which Cas9, an enzyme capable of recognizing specific base sequences and cleaving genes, can recognize single guide RNA (sgRNA) including those base sequences and cleave specific DNA.

Cas9 that recognizes sgRNA binds to a site located 3 base pairs upstream of a specific PAM, causing a double-stranded break in the DNA, and the DNA thus cleaved is repaired by two main mechanisms: homology-directed repair (HDR), which is based on the recombination of homologous sequences, and non-homologous end joining (NHEJ), which joins two ends regardless of sequence homology.

By making good use of these repair mechanisms, genome correction can be performed strategically, and one of the factors that has the greatest impact on genome correction is the sequence of the guide RNA, as the efficiency of gene correction can be determined depending on which sequence is used.

### DISCLOSURE

### Technical Problem

Thus, the present inventors discovered and selected guide RNAs suitable for transdifferentiated neural stem cells derived from a Huntington's disease patient having mutations, and corrected the transdifferentiated neural stem cells using CRISPR Cas9 technology, thereby completing a neural stem cell therapeutic agent for Huntington's disease.

Therefore, an object of the present invention is to provide a composition for correcting the huntingtin gene, including a guide RNA including a nucleotide sequence complementary to the 5′ untranslated region (UTR) and first exon of the huntingtin (HTT) gene, or DNA encoding the guide RNA; and a Cas protein or a polynucleotide encoding the same.

Another object of the present invention is to provide a method for correcting the huntingtin gene, the method including transducing a guide RNA including a nucleotide sequence complementary to the 5′ untranslated region (UTR) and first exon of the huntingtin (HTT) gene, or DNA encoding the guide RNA; and a polynucleotide encoding a Cas protein into induced neural stem cells (HD-ciNSCs) including a mutant huntingtin gene.

Still another object of the present invention is to provide a pharmaceutical composition for preventing or treating Huntington's disease, including the corrected induced neural stem cells (ciNSCs).

### Technical Solution

To achieve the above-described objects, the present invention provides a composition for correcting the huntingtin gene, including a guide RNA including a nucleotide sequence complementary to the 5′ untranslated region (UTR) and first exon of the huntingtin (HTT) gene, or DNA encoding the guide RNA; and a Cas protein or a polynucleotide encoding the same.

In one aspect of the present invention, the 5′ untranslated region (UTR) and first exon of the huntingtin (HTT) gene consist of a nucleotide sequence of SEQ ID NO: 1.

In one aspect of the present invention, the target sequence of the guide RNA includes a sequence of 5′-Nx-NGG-3 or 5-PAM(CCN)-Nx-3, wherein N is any one of A, G, C, and T, N in Nx is any one of A, G, C, and T, and x is a number within 20.

In one aspect of the present invention, the target sequence of the guide RNA includes a nucleotide sequence of SEQ ID NO: 2.

Further, the present invention provides a method for correcting the huntingtin gene, the method including transducing a guide RNA including a nucleotide sequence complementary to the 5′ untranslated region (UTR) and first exon of the huntingtin (HTT) gene, or DNA encoding the guide RNA; and a polynucleotide encoding a Cas protein into induced neural stem cells (HD-ciNSCs) including a mutant huntingtin gene.

In one aspect of the present invention, the mutant huntingtin gene includes an abnormal CAG repeat sequence in the first exon.

In one aspect of the present invention, the induced neural stem cells are induced from somatic cells of a Huntington's disease patient through a transdifferentiation process.

In addition, the present invention provides a pharmaceutical composition for preventing or treating Huntington's disease, including the corrected induced neural stem cells (ciNSCs).

Hereinafter, the present invention will be described in detail.

In one aspect, the present invention relates to a composition for correcting the huntingtin gene, including a guide RNA (sgRNA) including a nucleotide sequence complementary to the 5′ untranslated region (UTR) and first exon of the huntingtin (HTT) gene, or DNA encoding the guide RNA; and a Cas protein or a polynucleotide encoding the same.

The huntingtin (HTT) gene is located on chromosome 4 and includes 67 exons and 66 introns. Huntington's disease includes an abnormal CAG repeat sequence in the first exon region of the huntingtin gene, and is caused by the increased expression of the abnormal huntingtin protein. In the present invention, the composition is intended to correct the abnormal CAG repeat sequence included in the first exon of the huntingtin gene, and includes an sgRNA that targets the sequence.

As used herein, the term ‘guide RNA’ refers to RNA which is specific for target DNA, and serves to form a complex with a Cas protein and bring the Cas protein to the target DNA.

The guide RNA of the present invention may be single-guide RNA (sgRNA), and the sgRNA may be delivered to a cell or organism in the form of RNA or DNA encoding the sgRNA. The sgRNA may also be in the form of isolated RNA, RNA included in a viral vector, or RNA encoded by a vector. Preferably, the vector may be a viral vector or a plasmid, but is not limited to these types.

The guide RNA includes a nucleotide sequence complementary to the 5′ untranslated region (UTR) and first exon of the huntingtin (HTT) gene, which includes the nucleotide sequence of SEQ ID NO: 1. A nucleotide sequence targeted by the guide RNA includes a sequence 5′-Nx-NGG-3 or 5-PAM(CCN)-Nx-3, wherein N is any one of A, G, C, and T, N in Nx is any one of A, G, C, and T, and x is a number within 20. More specifically, the target sequence of the guide RNA may include a nucleotide sequence (RG1) of SEQ ID NO: 2, and the nucleotide sequence of SEQ ID NO: 2 is located simultaneously in the 5′ untranslated region (UTR) and first exon of the huntingtin (HTT) gene, and includes an abnormal CAG repeat sequence.

As used herein, the term ‘Cas protein’ refers to an essential protein element in the CRISPR/Cas system, and forms an active endonuclease or nickase when complexed with two RNAs called CRISPR RNA (crRNA) and trans-activating crRNA (tracrRNA), and serves to degrade double-stranded DNA. The Cas protein may preferably be a Cas9 protein.

In another aspect, the present invention relates to a method for correcting the huntingtin gene, the method including transducing a guide RNA including a nucleotide sequence complementary to the 5′ untranslated region (UTR) and first exon of the huntingtin (HTT) gene, or DNA encoding the guide RNA; and a polynucleotide encoding a Cas protein into induced neural stem cells (HD-ciNSCs) including a mutant huntingtin gene.

As used herein, the “neural stem cells” are undifferentiated cells that have the ability to self-renew and have the multipotency to differentiate into neurons and/or glia, for example, astrocytes, oligodendrocytes, and/or Schwann cells, and the like. Neural stem cells differentiate into neural cells, for example, neurons or glia, through the stages of neural progenitor cells or glial progenitor cells that give rise to specific neural cell types.

In the present invention, the ‘induced neural stem cells (ciNSCs)’ may be produced by ‘transdifferentiation’ of somatic cells from a Huntington's disease patient. The ‘transdifferentiation’ is a cell reprogramming technique that directly reprograms already mature differentiated cells back into stem cells, and may introduce exogenous genes through various methods, and although the method is not limited, preferably, direct conversion may be induced using small molecule compounds.

The ‘transdifferentiation’ is not limited to the method, but may include culturing human fibroblasts in a medium including a combination of one or more small molecule compounds of thiazovivin, valproic acid, purmorphamine, A8301, SB431542, deazaneplanocin A (DZNep), 5-AZA, and CHIR99021, and the human fibroblasts may be cultured by additionally including one or more of Sendai virus, mRNA, or miRNA in the medium.

Furthermore, the ‘induced neural stem cells including a mutant huntingtin gene (HD ciNSCs)’ are derived from the somatic cells of a Huntington's disease patient and include an abnormal CAG repeat sequence in the first exon of the huntingtin gene, and in an embodiment of the present invention, the induced neural stem cells derived from the cells of a Huntington's disease patient are referred to as ‘pre-corrected HD ciNSCs.’ In the present invention, the pre-corrected HD ciNSCs may be corrected by transducing a guide RNA including a nucleotide sequence complementary to the 5′ untranslated region (UTR) and first exon (exon) of the huntingtin (HTT) gene, or DNA encoding the guide RNA; and a polynucleotide encoding a Cas protein, and in an embodiment of the present invention, these are referred to as corrected ciNSCs.

In the present specification, the “transduction” is not limited to the method, and may be achieved by various methods known in the art, such as microinjection, electroporation, DEAE-dextran treatment, lipofection, nanoparticle-mediated transfection, protein transduction domain-mediated introduction, a viral or non-viral mediated gene delivery vehicle, and exosomes, and in an embodiment of the present invention, plasmid DNA was introduced using electroporation.

In addition, GABAergic neurons secrete GABA in the brain, and γ-aminobutyric acid (GABA) is a non-protein amino acid distributed in nature and is known as a neurotransmitter present in the brain and spinal cord of mammals, and is known to be lost in patients with Huntington's disease. The pre-corrected induced neural stem cells (HD ciNSCs) of the present invention showed much higher differentiation efficiency when induced to differentiate into GABAergic neurons lost in an actual Huntington's disease patient through correction.

In an embodiment of the present invention, somatic cells of a patient including a mutant huntingtin gene were first transdifferentiated into induced neural stem cells, the induced neural stem cells were transduced with a total of two recombinant plasmids: pRGEN_Cas9_CMV for encoding a Cas9 protein and pRGEN_Human_HTT encoding the sgRNA targeting the mutant HTT, and in this case, the Cas9 protein may cleave the target sequence region under the mediation of the sgRNA to form a DNA double-strand break, and trigger the self-damage repair mechanism of an organism to insert or delete a partial sequence. In the case of the present invention, the self-damage repair mechanism is used to block the expression of a mutant huntingtin (HTT) gene including abnormal CAG, thereby blocking the synthesis of a mutant HTT protein.

In another aspect of the present invention, the present invention relates to a pharmaceutical composition for preventing or treating Huntington's disease, including the corrected induced neural stem cells (ciNSCs).

As used herein, the term “prevention” refers to all actions that can suppress symptoms of Huntington's disease or delay the onset of the disease by administering the pharmaceutical composition according to the present invention.

As used herein, the term “treatment” refers to all actions that ameliorate or beneficially change symptoms of Huntington's disease by administering the pharmaceutical composition according to the present invention.

The pharmaceutical composition according to the present invention may be used after being formulated in the form of an oral formulation, such as a powder, a granule, a pill, a capsule, a suspension, an emulsion, a syrup, and an aerosol, an external preparation, a suppository, and a sterile injection solution, according to a typical method, and may further include a carrier, an excipient, or the like required for formulation. Examples of pharmaceutically acceptable carriers, excipients or diluents which may be further included in the active ingredient include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, and the like. When the pharmaceutical composition is formulated, the pharmaceutical composition is prepared using a diluent or excipient, such as a filler, an extender, a binder, a wetting agent, a disintegrant, and a surfactant, which are commonly used.

The pharmaceutical composition of the present invention can be administered by direct implantation or transfer to a desired tissue site to regenerate or functionally restore the damaged nervous system. For example, the neural stem cells of the present invention are transplanted directly into a damaged nerve site. Transplantation may be performed using a single cell suspension or an aggregate with a fixed cell density.

A suitable dose of the pharmaceutical composition of the present invention may vary depending on factors, such as formulation method, administration method, age, body weight, gender or disease condition of the patient, diet, administration time, administration route, excretion rate, and response sensitivity.

### Advantageous Effects

The present invention relates to a composition for gene correction for correcting a mutation in the huntingtin (HTT) gene using a CRISPR Cas9 system, a method for correcting the gene, and a pharmaceutical composition for preventing or treating Huntington's disease using the same, and creates transdifferentiated neural stem cells that can be mass-proliferated without gene insertion into the chromosomal DNA of somatic cells using a combination of small molecule compounds, and creates a cell therapeutic agent for Huntington's disease using the CRISPR Cas9 system to correct an excessive CAG repeat in the HTT gene responsible for Huntington's disease to a gene with a normal range of CAG repeats. That is, through transdifferentiation using small molecule compounds, it is possible to induce safe and high-quality neural stem cells derived from somatic cells of a Huntington's disease patient without gene insertion, and these neural stem cells can be used as a cell therapeutic agent for Huntington's disease using neural stem cells from which the cause of Huntington's disease has been removed through gene correction.

### DESCRIPTION OF DRAWINGS

FIG. 1 shows the candidate sgRNA locations for gene correction and the principle of gene correction using induced neural stem cells obtained from the somatic cells of a Huntington's disease patient. The above 5′ untranslated region and exon 1 sequence is SEQ ID No. 13, and the complementary sequence thereof is SEQ ID No. 14.

FIG. 2 is a schematic view showing the process of cloning and PCR screening of gene-corrected induced neural stem cells.

FIG. 3 is a view showing a method for selecting corrected induced neural stem cells through T7E1 assay after gene correction.

FIG. 4 shows the results of confirming gene-corrected sites by PCR using clones of induced neural stem cells selected through the process shown in FIG. 3.

FIG. 5 shows the results of PCR in FIG. 4, confirming the expression of a mutant HTT (mHTT) having mutant CAG repeat sequences through western blot.

FIG. 6 shows the results of confirming sequence changes (indels) for the target position for three off-target candidates using band PCR and whole genome sequencing as a 3-base pair mismatch off-target candidate identified by a RGEN tool. The sequence in FIG. 6 is SEQ ID NO: 15.

FIG. 7 shows the results of differentiation of neural stem cells derived from somatic cells of a Huntington's disease patient before and after gene correction into GABAergic neurons, which are known to be lost in Huntington's disease, and the differentiation was confirmed using immunostaining.

FIG. 8 shows the results of a strategic analysis of GABAergic neurons before and after gene correction, as confirmed by immunofluorescence staining in FIG. 7.

FIG. 9 shows the results of confirming the relative expression levels of GABAergic neuron markers GAD65, GABRA1, and GABRA2 using real-time PCR.

FIG. 10 shows the results of comparing the expression levels of apoptosis-related genes (caspase-3, p53, and annexin V) between neural stem cells derived from a Huntington's disease patient and gene-corrected neural stem cells.

FIG. 11 shows the results of comparing cell proliferation rates between neural stem cells derived from a Huntington's disease patient and gene-corrected neural stem cells.

FIG. 12 shows the results of comparing the formation of neurospheres in relation to apoptosis and proliferation rates between neural stem cells derived from a Huntington's disease patient and gene-corrected neural stem cells.

FIG. 13 shows the results of comparing the expression of an apoptosis-related gene (caspase-3) between neural stem cells derived from a Huntington's disease patient and gene-corrected neural stem cells through immunostaining.

FIG. 14 is a schematic view of an experiment for confirming the recovery of motor ability by administering gene-corrected neural stem cells using an animal model of Huntington's disease created by intracerebral injection of a toxin (quinolinic acid; QA).

FIG. 15 shows the recovery of motor ability by administering gene-corrected neural stem cells to an animal model of Huntington's disease created by intracerebral injection of a toxin (quinolinic acid; QA), as confirmed by the experimental results in FIG. 14.

FIG. 16 is a schematic view of an experiment for confirming the recovery of motor ability by administering gene-corrected neural stem cells using a R6/2 model, which is a Huntington's disease transgenic mouse.

FIG. 17 shows the results of motor ability recovery by administering gene-corrected neural stem cells using the R6/2 model, as confirmed by the experimental results in FIG. 16.

FIG. 18 shows the results of measuring the survival rate and body weight change by administering gene-corrected neural stem cells using a R6/2 model, which is a Huntington's disease transgenic mouse.

FIG. 19 shows the comparison of motor ability recovery degrees in order to verify the effectiveness before and after gene correction by injecting pre-corrected neural stem cells (HD ciNSCs) and corrected neural stem cells (ciNSCs) into a R6/2 model, which is a Huntington's disease transgenic mouse.

FIG. 20 shows the results of performing immunohistochemistry using brain tissues around the injection sites in the neural stem cell injection groups.

### BEST MODE

Hereinafter, the present specification will be described in detail with reference to examples in order to specifically explain the present specification. However, the examples according to the present specification may be modified into various forms, and it should not be interpreted that the scope of the present specification is limited to the examples described below in detail. The examples of the present specification are provided to more completely describe the present specification to a person with ordinary skill in the art.

<Example 1> Selection of sgRNA Target Site for CAG Correction of Human HTT Gene

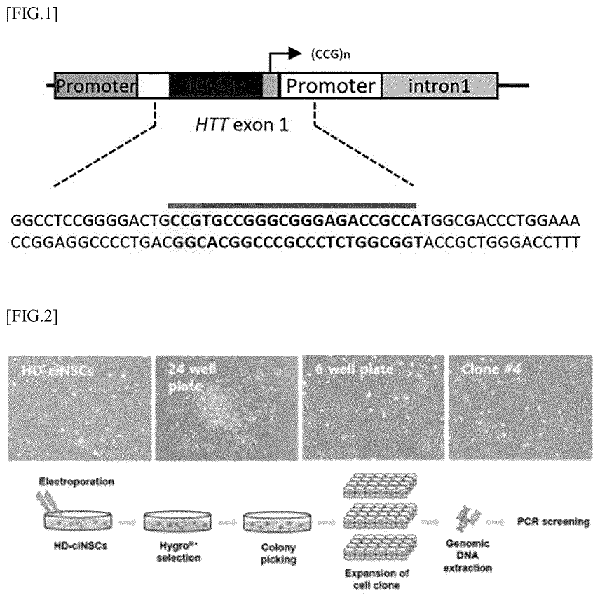

The HTT gene is located on chromosome 4 and includes 67 exons and 66 introns. An abnormal CAG repeat sequence to be corrected in the HTT gene is located in the first exon, and the sgRNA target sites selected for correction are located in the 5′ untranslated region and first exon (FIG. 1). The above 5′ untranslated region and exon 1 sequence are designated as SEQ ID No. 13, and the complementary sequence thereof is designated as SEQ ID No. 14.

In the human genome, the nucleotide sequence of the first exon, including the 5′ untranslated region and the conserved region of the HTT gene is identical to SEQ ID NO: 1. Positions 1-315 of SEQ ID NO: 1 are the positions of the 5′ untranslated region, and positions 316-615 are the positions of the first exon.

In the present invention, one strand of the double-stranded DNA targeted by CRISPR/Cas9 has the following structure: 5-Nx-NGG-3 or 5-PAM(CCN)-Nx-3, where N is any one of A, G, C, and T, N in Nx is any one of A, G, C, and T, and x is 20. Specifically, the target sequence includes a nucleotide sequence of the following SEQ ID NO: 2, and the target (RG1) is located simultaneously in the 5′ untranslated region and first exon.

SEQ ID NO: 2: RG1:
5′-CCGTGCCGGGCGGGAGACCGCCA-3′

<Example 2> Transdifferentiation of Somatic Cells into Induced Neural Stem Cells (HD-ciNSCs)

Somatic cells (fibroblasts) including a mutation-induced huntingtin (HTT) gene were transdifferentiated into induced neural stem cells (HD ciNSCs). The fibroblasts were obtained from a Huntington's disease patient.

Specifically, 1×10⁵ fibroblasts were prepared in a 60 mm dish, and the prepared cells were cultured by adding eight small molecule compounds related to reprogramming (thiazovivin, valproic acid, purmorphamine, A8301, SB431542, CHIR99021, 5-aza-2′-deoxytidine, and DZNep) to a neurobasal medium consisting of DMEM/F12, N2 supplement, B27 supplement, bFGF, and EGF. The characteristics and differentiation method of each of the eight small molecule compounds are as described in a prior patent (Korean Patent No. 10-1816103).

### <Example 3> Transduction of Induced Neural Stem Cells

The induced neural stem cells (HD ciNSCs) prepared in 1-2 above were transduced for gene correction using the CRISPR/Cas9 method.

First, to express the Cas9 protein, a recombinant plasmid pRGEN_Cas9_CMV (7383 bp) including a CMV promoter, and a recombinant plasmid pRGEN_Human_HTT (2488 bp), which can be used to transcribe sgRNA for the target selected in 1-1 above, were each constructed. The two recombinant plasmids (pRGEN_Cas9_CMV, pRGEN_Human_HTT) were added to ep-tubes at 1 μg and 500 ng, respectively, in a ratio of 2:1 at a concentration of 1.5 μg/ul, and pipetted to mix well.

The patient-derived neural stem cells (HD ciNSCs) including the mutant huntingtin gene in 1-2 above were washed twice with 3 ml of PBS after removing the culture medium. 1 ml of StemPro Accutase™ Cell Dissociation Reagent (Gibco) was added to the HD-ciNSCs to detach the cells, and the cells were allowed to react in a CO₂ incubator at 37° C. for 3 minutes. After the reaction, when it was confirmed that the cells had been detached by tapping the side of the dish 5 to 6 times, the dish was wiped after adding 2 ml of a ciNSC culture medium, and then a total of 3 ml was transferred to a 15 ml tube. The transferred cells in the 15 ml tube were counted and centrifuged at 1500 RPM for 3 minutes. After a cell pellet was confirmed, only the supernatant was removed while paying close attention to the pellet. Resuspension Buffer R was added to the cell pellet at a concentration of 4×10⁵ cells/10 μl, and the cell pellet was resuspended by pipetting. After PLO/FN coating, 2 ml of ciNSC culture medium without pen/strep was dispensed into each well of a 6-well plate fully coated with PLO/FN, and the culture medium was warmed in a humidified 37° C./5% CO₂ incubator.

Thereafter, the prepared recombinant plasmids (pRGEN_Cas9_CMV, pRGEN_Human_HTT) and the cell R buffer suspension were thoroughly mixed, and then the plasmids were transfected into the cells according to the Neon electroporation system (Thermofisher Scientific) protocol provided by the manufacturer (1400 V, 20 ms, 1 time).

Thereafter, the cultured cells were detached using the same method and re-cultured at 1×10³ cells in 100 mm dishes for 8 days, and then the formed colonies were cultured in a 24-well plate and cloned.

### <Experimental Example 1> Confirmation of HTT Gene Correction by PCR

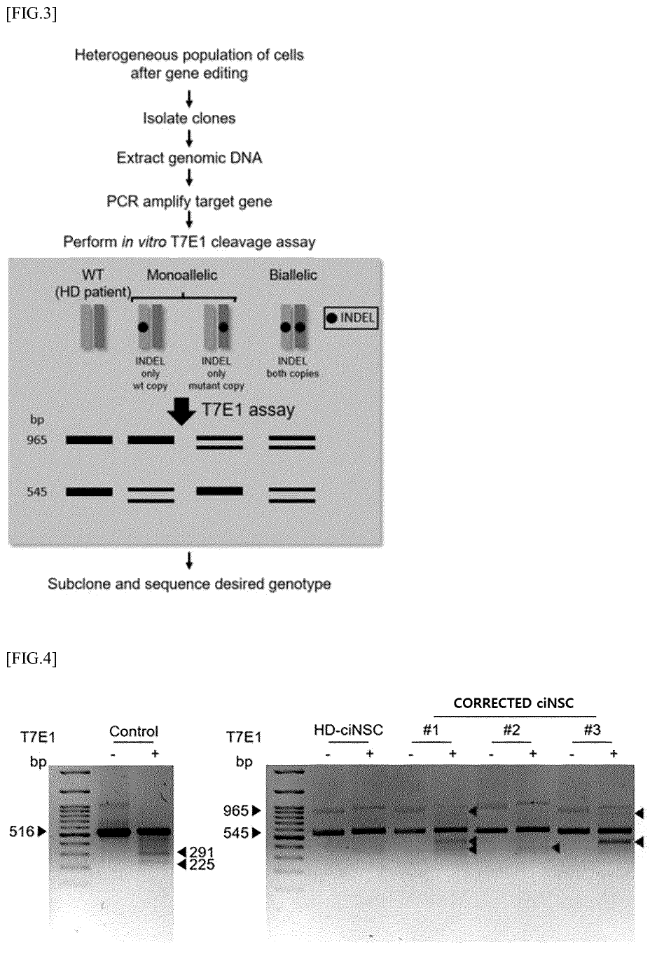

According to this example, PCR was performed to confirm the effect of correcting the mutant HTT gene, and the experimental process is shown in FIG. 3. The cells cloned in the above example were cultured in a 6-well plate and detached with Accutase when confluency reached 70 to 80%, and genomic DNA was obtained using a QIAamp DNA Mini Kit (QIAGEN). The obtained DNA was subjected to PCR using PrimeSTAR GXL DNA polymerase (TAKARA) and CAG repeat primers of SEQ ID NOS: 3 and 4. The PCR was carried out under the conditions of (98° C.-10 s, 68° C.-30 s) for a total of 30 cycles, and a PCR product band was confirmed by electrophoresis on a 1.6% agarose gel.

TABLE 1

| | | |
|---|---|---|
| Forward | 5′-TGC CGG GAC GGG | SEQ ID NO: 3 |
| primer | TCC AAG ATG-3′ | |
| Reverse | 5′-TGG GTT GCT GGG | SEQ ID NO: 4 |
| primer | TCA CTC TGT C-3′ | |

In healthy individuals, a band of 500 to 600 bp is detected, while in Huntington's disease patients, a band between 600 and 1000 bp is confirmed depending on the number of CAG repeats. To confirm the genotyping of gene correction, gene correction was analyzed by performing genotyping using the AccuCRISPR™ Mutation Detection Kit (Bioneer) according to the protocol provided by the manufacturer. As a result, as shown in FIG. 4, it was confirmed that a mutant CAG repeat (150 times) band was finally cleaved by T7 in #1 and #3.

### <Experimental Example 2> Confirmation of Huntingtin Protein Expression by Western Blot

The cloned cells during culture in Example 3 were resuspended using RIPA Lysis and Extraction Buffer (Thermofisher Scientific) and Pierce Protease inhibitor (Thermofisher Scientific), and proteins were extracted according to the manufacturer's protocol.

Protein concentration was measured using the Bradford assay, and the final protein concentration was prepared at 2 μg/ml using Pierce™ Lane Marker Non-Reducing Sample Buffer (Thermofisher Scientific) and tertiary distilled water, and the protein was denatured in boiling water for 5 minutes. Thereafter, the proteins were electrophoresed using 4% to 15% TGX gels (Bio-Rad) and Tris-glycine-SDS (TGS) running buffer (Bio-Rad), and transferred to PVDF membranes (Bio-Rad). Thereafter, 0.1% Tween 20 and 5% skim milk were added to PBS, and the resulting mixture was blocked at room temperature for 1 hour. Thereafter, a primary antibody (Total HTT, MAB2166) was mixed with the solution of 0.1% Tween 20 and 5% skim milk in PBS at a ratio of 1:1000, the resulting mixture was incubated at 4° C. for 16 hours, a secondary antibody (Goat anti-Mouse IgG (H+L) Secondary Antibody, 31430, Thermofisher Scientific) was mixed with the solution of 0.1% Tween 20 and 5% skim milk in PBS at a ratio of 1:10000, the resulting mixture was incubated at room temperature for 1 hour (ECB), and the expression of the mutant huntingtin protein was confirmed using ECL buffer (Bio-Rad).

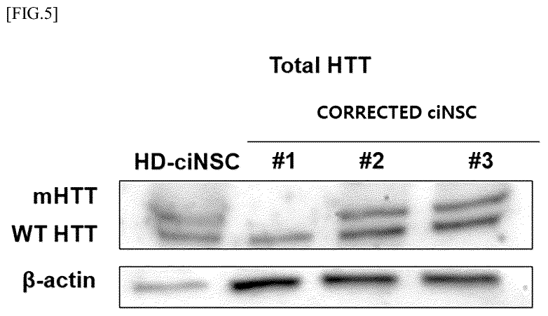

As a result, as shown in FIG. 5, the expression of mutant HTT (mHTT) with a mutant CAG repeat was confirmed by western blot, and finally, it was confirmed that the gene was corrected because mHTT was not expressed in lane #1.

### <Experimental Example 3> Off-Target Effect Analysis

### 3-1. Whole Genome Sequencing

Sequencing libraries were prepared according to the manufacturer's instructions for the TruSeq PCR-free DNA High Throughput Library Prep Kit (Illumina). Briefly, 1 μg of genomic DNA was sheared and fragmented using adaptive focused acoustic technology (Covaris). Thereafter, the end of the fragmented DNA was repaired to generate 5′-phosphorylated blunt-ended dsDNA. After the repair, the DNA was selected using a bead-based method. These DNA fragments were ligated with TruSeq DNA UD indexing adapters by adding a single ‘A’ base.

The purified libraries were quantified using qPCR according to the qPCR Quantification Protocol Guide (Illumina Sequencing Platforms for KAPA Library Quantification kit) and quantified using a High-sensitivity DNA chip (Agilent Technologies). Thereafter, paired-end sequencing (2×150 bp) was performed at Macrogen using Novaseq (Illumina).

### 3-2. Confirmation of Off-Target Effect Using PCR

The sgRNA (SEQ ID NO:15) used in the present invention was confirmed to have 3-bp mismatches on chromosomes 1 (chr1), 19 (chr19), and 20 (chr20) using the RGEN tool, these were considered to be 3-base pair mismatch off-target candidates, and the off-target effect for the same was verified by PCR. When an intact single band was formed by performing PCR, it was determined that no frameshift had occurred.

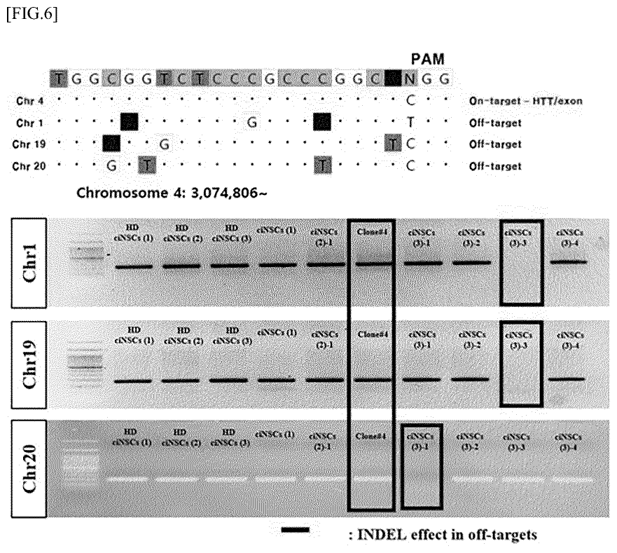

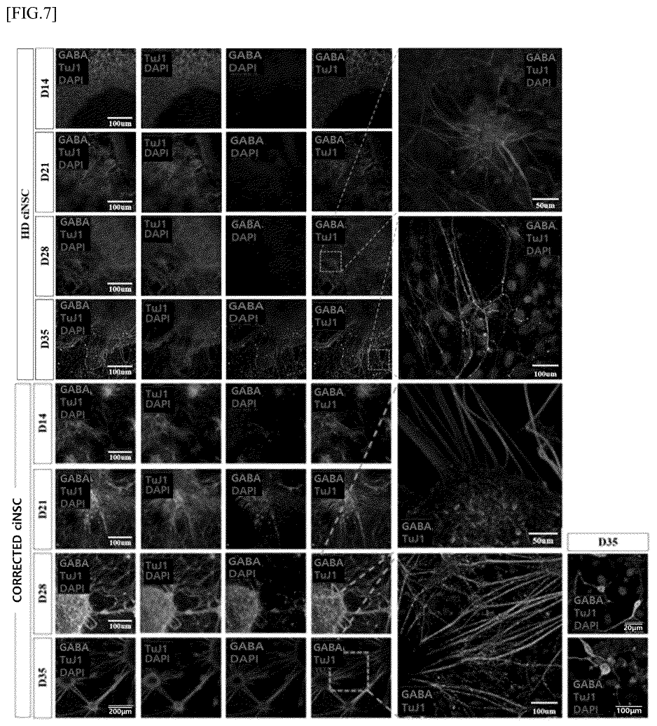

The primer sequences used in the experiment are shown in the following table (SEQ ID NO: 5 to SEQ ID NO: 12). PCR was performed using Easytaq (TransGen Biotech) and confirmed by electrophoresis on a 1.6% agarose gel. As a result, as shown in FIG. 6, the selected sgRNA did not show any changes (indels) in the three off-target candidates in clone #4, confirming there was no off-target effect.

TABLE 2

| | | | |
|---|---|---|---|
| Chr1 | off-target_ | 5′-CAG CAC AGA | SEQ ID |
| | Chr1_F | ACT GCA GAC AGC-3′ | NO: 5 |
| | off-target_ | 5′-AGA CCC AAG | SEQ ID |
| | Chr1_R | ACA CAG GAC AGC-3′ | NO: 6 |
| Chr19 | Off-target_ | 5′-TAG ACG AGG | SEQ ID |
| | Chr19_F | TTG GGG CTG C-3′ | NO: 7 |
| | Off-target_ | 5′-ACG ACT CGT | SEQ ID |
| | Chr19_R | TCA CGC CTG C-3′ | NO: 8 |
| Chr20 | Off-target_ | 5′-CCA ATG TCT | SEQ ID |
| (1^{st} | Chr20_1^{st}_F | GTC CAC AGC-3′ | NO: 9 |
| PCR) | Off-target_ | 5′-TTC ACG CCA | SEQ ID |
| | Chr20_1^{st}_R | TTC TCC TGC-3′ | NO: 10 |
| Chr20 | Off-target_ | 5′-TGG GAG TCT | SEQ ID |
| (Nested | Chr20_2^{nd}_F | CAC TCT GTT GC-3′ | NO: 11 |
| PCR; 2^{nd} | Off-target_ | 5′-CTG GGT TTC | SEQ ID |
| PCR) | Chr20_2^{nd}_R | ACC GTG TTA GC-3′ | NO: 12 |

### <Experimental Example 4> Results of GABAergic Neuron Differentiation

### 4-1. Immunofluorescence Staining

Neural stem cells derived from somatic cells of a Huntington's disease patient before and after gene correction were differentiated into GABAergic neurons. Pre-corrected HD ciNSCs and corrected ciNSCs were seeded at 5×10⁴ cells/well in a 24-well plate coated with poly-L-ornithine/fibronectin (PLO/FN) the day before treatment with a GABA-inducing culture medium. For 0 to 7 days, the cells were cultured in a culture medium in which DMEM F12 and Neurobasal media were mixed at a ratio of 1:1, supplemented with a culture medium including 0.5× N2 supplement, 0.5× B27 supplement, 1× MEM-NEAA, 100 μg/ml penicillin and streptomycin, 10 μM valproic acid, 0.65 μM purmorphamine, 2 μM SB431542, 0.1 μM retinoic acid, 1 μM CHIR99021, 200 nM dorsomorphin, 100 nM LDN193189, and 200 μM ascorbic acid.

Thereafter, GABAergic neurons were matured while being cultured for 7 to 35 days in a culture medium in which DMEM F12 and neurobasal media were mixed at a ratio of 1:1, supplemented with 0.5× N2 supplement, 0.5× B27 supplement, 1× MEM-NEAA, 100 g/ml penicillin and streptomycin, and 200 μM ascorbic acid. Neuronal-like GABAergic neurons were confirmed by immunocytostaining on days 14, 21, 28, and 35 after culture.

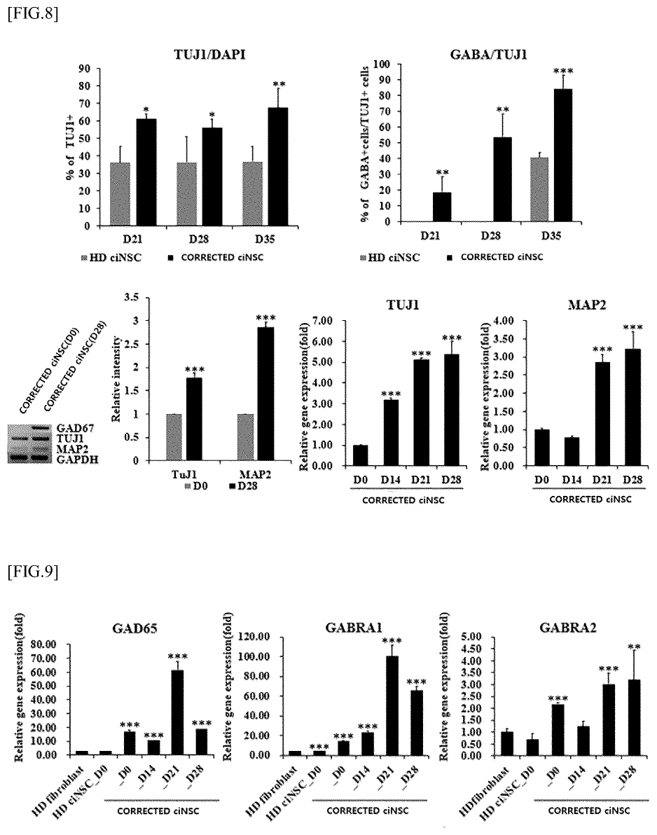

As a result, as shown in FIG. 7, it was confirmed that the corrected ciNSCs had a higher differentiation rate into neurons and a higher differentiation rate into GABAergic neurons than the pre-corrected HD ciNSCs. Furthermore, MAP2, a mature neuron marker, was not confirmed in Huntington's disease patient cells until day 28. The aforementioned results were quantitatively confirmed as shown in FIG. 8.

### 4-2. Expression of Genetic Markers

Each RNA was collected using Trizol (Thermo Fisher) on days 14, 21, and 28 when the corrected ciNSCs began to differentiate into GABAergic neurons. The recovered RNA was converted to cDNA by performing RT-PCR. The relative expression levels of GABAergic neuron markers GAD65, GABRA1, and GABRA2 in the cDNA were confirmed using real-time PCR using SYBR green (KAPA). As a result, as shown in FIG. 9, it was confirmed that the expression of GABAergic neuron marker genes increased as differentiation progressed. Further, it was confirmed that the ability to suppress gene expression varied depending on the concentration, and expression was stably suppressed at 400 pmol.

### <Experimental Example 5> Comparison of Apoptosis and Proliferation Rate

### 5-1. Reduced Apoptosis by Gene Correction

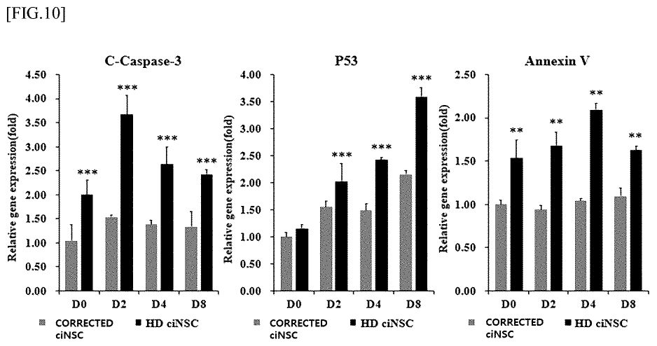

Neural stem cells have the property of clumping together if they are unable to adhere. Pre-corrected induced neural stem cells (HD ciNSCs) and corrected induced neural stem cells (ciNSCs) were cultured at a density of 1×10⁶ cells per 100 mm Petri dish for 4 days. Thereafter, as shown in FIG. 10, it could be confirmed that the expression of apoptosis-related genes caspase-3, p53, and annexin V all increased in each of the cultured cells.

### 5-2. Confirmation of Cell Proliferation Rate by Gene Correction

After 1×10⁵ cells were cultured on a 60 mm dish coated with PLO/FN for 4 days, the cell amount was measured to determine the cell proliferation rate. As a result, as shown in FIG. 11, it was confirmed that the doubling time of the pre-corrected HD ciNSCs was approximately 14 hours slower than that of the corrected ciNSCs.

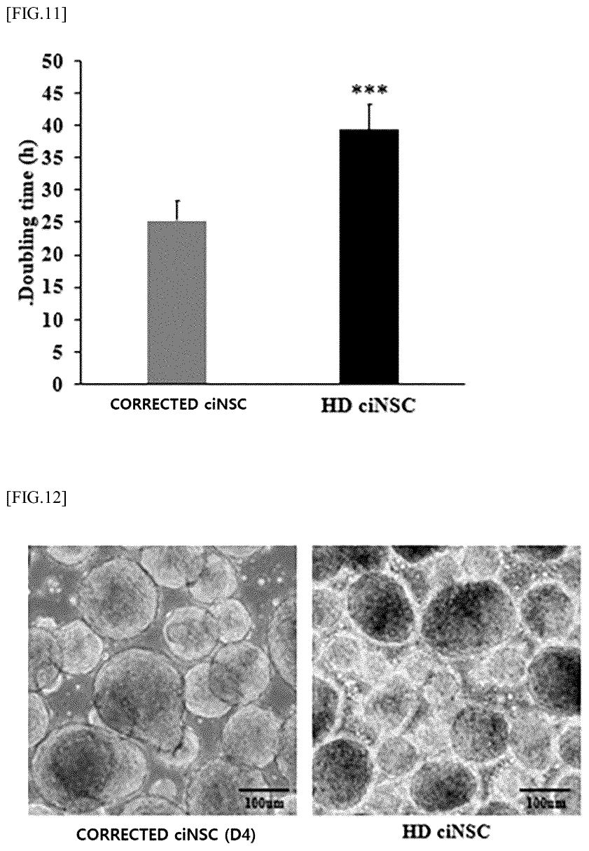

Furthermore, as a result of observing the neurospheres formed as a result of the culture under an optical microscope, it could be confirmed that the pre-corrected HD ciNSCs had a slow proliferation rate and frequent apoptosis, resulting in uneven and rough neurospheres, whereas the corrected ciNSCs formed smooth neurospheres (FIG. 12).

### 5-3. Comparison of Apoptotic Gene Expression During GABAergic Neuron Differentiation

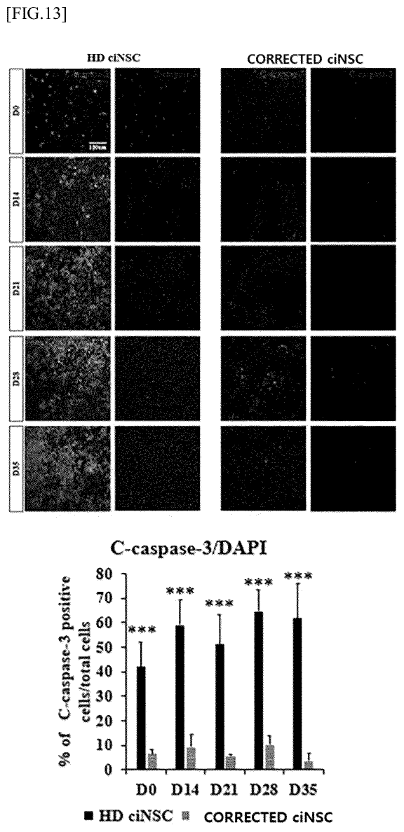

For the GABAergic neurons differentiated in Experimental Example 4, the expression of an apoptosis-related gene (caspase-3) was confirmed by the methods of 5-1 and 5-2 above. As a result, as shown in FIG. 13, it was confirmed that the pre-corrected HD ciNSCs showed higher apoptotic gene expression before differentiation compared to the corrected ciNSCs, and maintained the high expression even during differentiation into GABAergic neurons.

### <Experimental Example 6> Effect of Toxicity on Motor Function Recovery in Huntington's Disease-Induced Animals

To induce Huntington's disease in SD rats, quinolinic acid (QA) was dissolved in IN NaOH and then dissolved at 200 nmol/2 μl in sterile 0.1 M PBS (pH 7.4) and administered (AP: +0.8 mm, ML: +2.8 mm, DV: −5.5 mm).

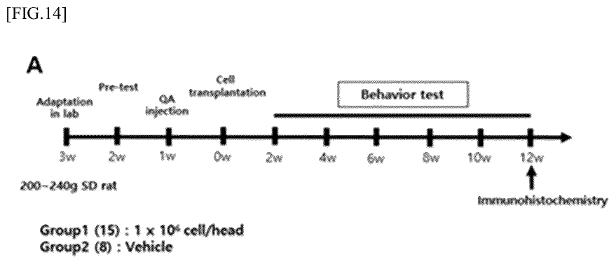

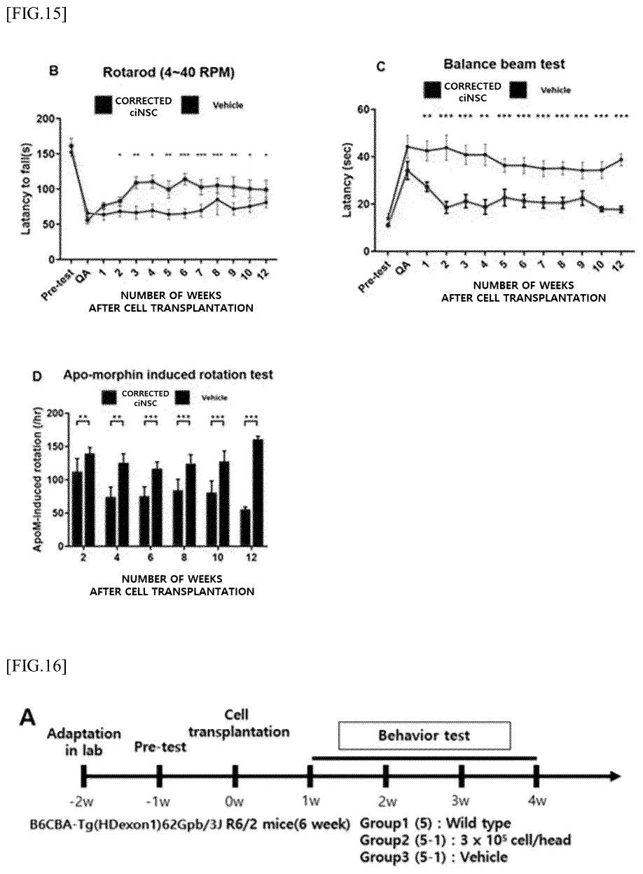

After cell transplantation, to eliminate xenogeneic immune responses, the immunosuppressive agent cyclosporin A was diluted in PBS at 10 mg/kg and administered intraperitoneally once daily from two days before neural stem cell administration until autopsy, and Huntington's disease autologous skin cell-derived gene-corrected neural stem cells (ciNSCs) (experimental group, G1) and a vehicle (control, G2) were each administered. For direct intrastriatal administration, stereotaxic equipment was used to administer them to the right position ML: +2.2 mm, AP: 0.2 mm, DV: −4.5 mm based on the bregma. The process is illustrated in FIG. 14.

After administration, no deaths or behavioral abnormalities due to the administration of test substances were observed in any of the groups (G1 and G2).

### 6-1. Rotarod Test

All animals to be used in the experiment were trained for 8 days before QA administration and then 1 day before the measurement day. On the day of measurement, the experimental animal was placed on the center of the rotating rod of a rotarod. The rotarod was rotated at an accelerating rate of 4 to 40 rpm for 5 minutes, and the time (sec) from the start of rotation to the time when the experimental animal fell off was measured. The average of the values measured a total of three times was used as the measured value for each animal. The measurement was performed once a week before QA administration, and before and after test substance administration.

As a result of the rotarod test measurements, a statistically significant increase in fall time was observed from week 1 to week 12 in the corrected neural stem cell-treated group (G1) compared to the vehicle control (G2).

### 6-2. Balance Beam Test

All animals to be used in the experiment were trained for 5 days before QA administration and then 1 day before the measurement day. On the day of measurement, the experimental animal was placed on the first part of a beam. The time it took for the experimental animal to traverse a beam with a height of 100 cm, a length of 120 cm, and a width of 5 cm was measured. The measurement was performed once a week before QA administration, and before and after test substance administration.

As a result of the balance beam test measurement, a statistically significant decrease in beam transit time was observed from week 2 to week 12 in the corrected neural stem cell-treated group (G1) compared to the vehicle control (G2).

### 6-3. Apomorphine-Induced Rotation Test

1 mg/kg of apomorphine was subcutaneously injected into rats. Thereafter, the number of rotations in the rotation chamber was measured for 1 hour. The measurement was performed once two weeks before QA administration, and before and after test substance administration.

As a result of the test, a statistically significant decrease in number of rotations from 2 to 12 weeks was observed in the corrected neural stem cell-treated group (G1) compared to the vehicle control (G2).

As discussed above, in an animal model in which Huntington's disease was induced by toxicity, it could be confirmed that motor ability was recovered when gene-corrected neural stem cells (ciNSCs) were injected into the brain. (FIG. 15)

<Experimental Example 7> Effect of Recovering Motor Function in Animals with Huntington's Disease (R6/2)

The R6/2 mouse is a representative acute Huntington's disease model, and is a transgenic model of Huntington's disease, which includes exon 1 of human mutant Htt having 150 or more mutant CAG repeats. For administration, Huntington's disease autologous skin cell-derived gene-corrected neural stem cells were cultured in large quantities in 100 mm dishes. Before administration, a certain amount of the cells were isolated from the cell culture dish and stained with Trypan blue, and then viable cells were counted using an optical microscope and a hematocytometer, and the viability and viable cell concentration were calculated. When the viable cell concentration after cell counting is outside the reference range (existing concentration ±10%), the number of viable cells was corrected and then diluted with a vehicle to match the administration concentration.

After cell transplantation, to suppress xenogeneic immune responses, the immunosuppressive agent cyclosporin A was diluted in PBS at 10 mg/kg and administered intraperitoneally once a day from two days before test substance administration until autopsy. Thereafter, Huntington's disease autologous skin cell-derived gene-corrected neural stem cells (ciNSCs) (experimental group, G2) and vehicle (control, G3) were each administered. For direct intrastriatal administration, stereotaxic equipment was used to administer them bilaterally to both positions ML: 2.0 mm, AP: 0.0 mm, DV: −3.25 mm based on the bregma. The process is illustrated in FIG. 16.

In all animals, no behavioral abnormalities were observed due to the administration of corrected neural stem cells.

### 7-1. Rotarod Test

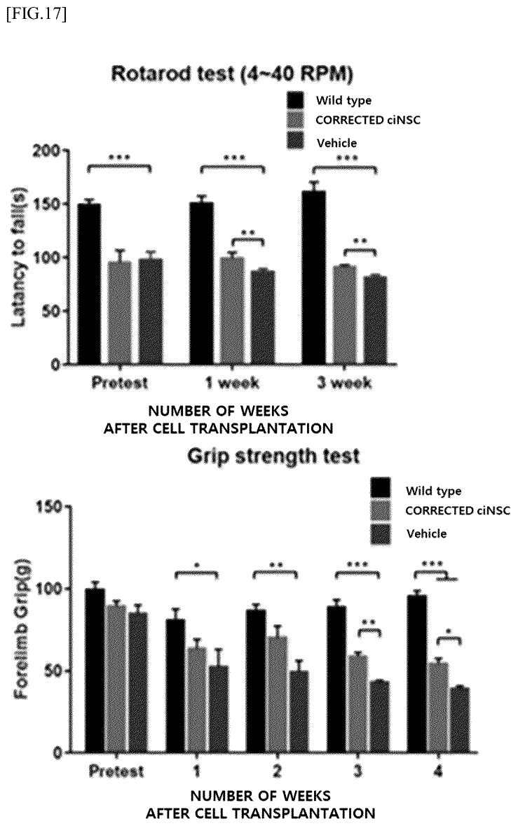

The test was performed once a week in the same manner as in 6-1 above. As a result, a statistically significant increase in fall time was observed from week 1 to week 3 in the corrected neural stem cell-administered group (G2) compared to the vehicle-administered group (G3) (FIG. 17).

### 7-2. Grip Strength Test

A grip strength test was performed on each of the experimental groups, and the grip strength of the mice in each experimental group was measured once a week when they gripped the grip and then dropped their front paws. As a result, a statistically significant increase in grip strength was observed from week 1 to week 4 in the corrected neural stem cell-administered group (G2) compared to the vehicle-administered group (G3) (FIG. 17).

### 7-3. Measurement of Body Weight and Survival

The body weights of all animals were measured every 3 days from the day of admission. Furthermore, the date of death of all animals was recorded.

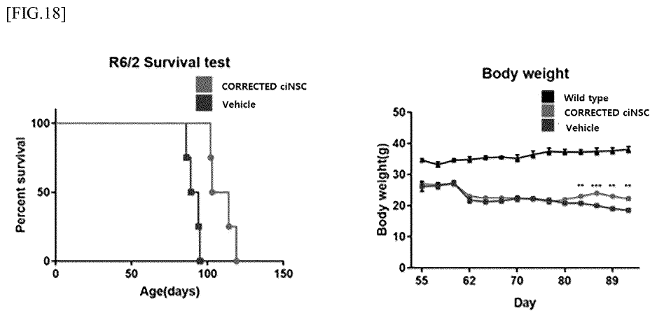

As a result, as shown in FIG. 18, it could be confirmed that the survival rate of the vehicle-treated group began to decrease after about 80 days, but the corrected neural stem cell-treated group (G2) had a longer survival period. Further, it was confirmed that the body weight loss in the corrected neural stem cell-administered group (G2) was reduced compared to the vehicle-administered group (G3) from 80 days of age.

### 7-4. Verification of Effectiveness of Gene Correction

To verify the effectiveness of neural stem cells derived from a Huntington's disease patient before and after gene correction, pre-corrected Huntington's disease autologous skin cell-derived neural stem cells (pre-corrected HD ciNSCs, G1) and corrected neural stem cells (corrected ciNSCs, G2) were each administered using the R6/2 mouse model in the same manner as described above, and experiments related to motor ability loss and maintenance were performed.

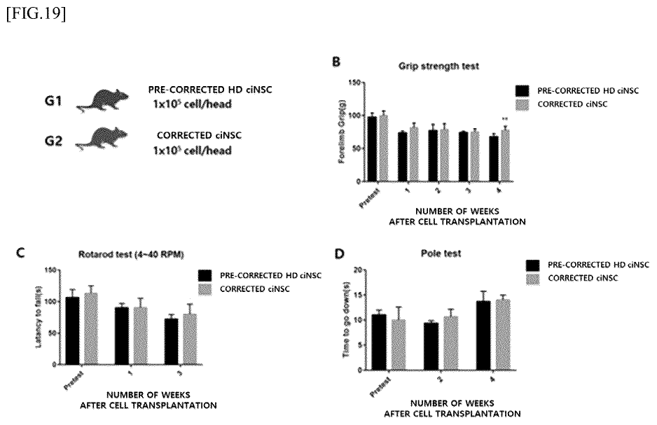

As a result, as shown in FIG. 19, it could be confirmed from the results of the grip strength test and the rotarod test that the corrected neural stem cells (corrected ciNSCs, G2) administered group showed a higher degree of recovery from motor ability loss than G1. In addition, even from the results of a pole test in which the ability of the mice to grasp a pole and move in order to descend into the cage was evaluated, in which the mice were placed on the top of the pole with their heads facing up, and the time it took for them to descend into their home cage was measured, it could be confirmed that the motor abilities described above were recovered in the G2-treated group.

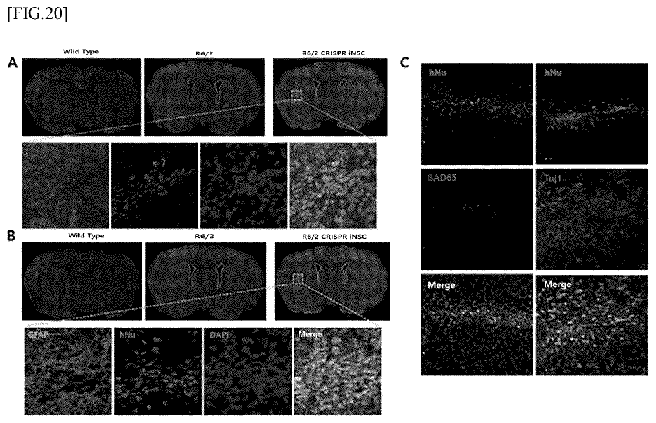

Furthermore, in immunohistochemistry using brain tissue surrounding the injection site in the neural stem cell administered group, staining was performed using human-specific markers (Human Nuclear, Human Mitochondria) and neuronal markers to confirm the survival of the transplanted neural stem cells in the brain. (FIG. 20)

From the above results, it could be confirmed that the correction of the mutant huntingtin gene was effective.

In the foregoing, the present invention has been examined mainly based on the preferred examples thereof. A person with ordinary skill in the art to which the present invention pertains will be able to understand that the present invention may be implemented in a modified form without departing from the essential characteristics of the present invention. Therefore, the disclosed examples should be considered illustrative rather than restrictive. The scope of the present invention is defined not in the above-described description, but in the claims, and it should be interpreted that all differences within a range equivalent thereto are included in the present invention.

### Modes of the Invention

In one aspect, the present invention relates to a composition for correcting the huntingtin (HTT) gene, including a guide RNA including a nucleotide sequence complementary to the 5′ untranslated region (UTR) and first exon of the huntingtin (HTT) gene, or DNA encoding the guide RNA; and a Cas protein or a polynucleotide encoding the same.

In one embodiment, the 5′ untranslated region (UTR) and first exon of the huntingtin (HTT) gene include a nucleotide sequence of SEQ ID NO: 1.

In one embodiment, the target sequence of the guide RNA includes a sequence of 5-Nx-NGG-3 or 5-PAM(CCN)-Nx-3 included in the nucleotide sequence of SEQ ID NO: 1, wherein N is any one of A, G, C, and T, N in Nx is any one of A, G, C, and T, and x is a number within 20.

In one embodiment, the target sequence of the guide RNA may include a nucleotide sequence of SEQ ID NO: 2.

In one embodiment, the target sequence of the guide RNA is a mutated huntingtin (HTT) gene including an abnormal CAG repeat sequence.

In one embodiment, the Cas protein is Cas9.

In another aspect, the present invention relates to a method for correcting the huntingtin gene, the method including transducing DNA encoding a guide RNA including a nucleotide sequence complementary to the 5′ untranslated region (UTR) and first exon of the huntingtin (HTT) gene; and a polynucleotide encoding a Cas protein into induced neural stem cells (HD-ciNSCs) including a mutated huntingtin gene including an abnormal CAG repeat sequence.

In one embodiment, the transduction may be performed using any one method of microinjection, electroporation, DEAE-dextran treatment, lipofection, nanoparticle-mediated transfection, protein transduction domain-mediated introduction, a viral or non-viral mediated gene delivery vehicle, and exosomes.

In one embodiment, the viral or non-viral mediated gene delivery vehicle may be a viral vector or a plasmid.

In one embodiment, induced neural stem cells (HD-ciNSCs) including the mutated huntingtin (HTT) gene may be induced to transdifferentiate from somatic cells of a Huntington's disease patient.

In one embodiment, the transdifferentiation may include culturing somatic cells from a Huntington's disease patient in a medium including one or more of thiazovivin, valproic acid, purmorphamine, A8301, SB431542, deazaneplanocin A (DZNep), 5-AZA, and CHIR99021 in combination.

In still another aspect of the present invention, the present invention relates to a pharmaceutical composition for preventing or treating Huntington's disease, including induced neural stem cells (ciNSCs) corrected by the aforementioned composition.

In one embodiment, the corrected induced neural stem cells (ciNSCs) may be induced neural stem cells (HD-ciNSCs) including a mutated huntingtin (HTT) gene including an abnormal CAG repeat sequence, wherein the nucleotide sequence of SEQ ID NO: 2 included in the 5′ untranslated region (UTR) and exon 1 of the ciNSCs has been corrected.

In yet another aspect of the present invention, the present invention relates to a method for correcting the huntingtin gene, the method including transducing DNA encoding a guide RNA including a nucleotide sequence complementary to the 5′ untranslated region (UTR) and first exon of the huntingtin (HTT) gene; and a polynucleotide encoding a Cas protein into induced neural stem cells (HD-ciNSCs) including a mutated huntingtin gene including an abnormal CAG repeat sequence to correct the huntingtin gene.

In one embodiment, induced neural stem cells (HD-ciNSCs) including the mutated huntingtin (HTT) gene are induced to transdifferentiate from somatic cells of a Huntington's disease patient.

In one embodiment, the transdifferentiation may include culturing somatic cells from a Huntington's disease patient in a medium including one or more of thiazovivin, valproic acid, purmorphamine, A8301, SB431542, deazaneplanocin A (DZNep), 5-AZA, and CHIR99021 in combination.

In one embodiment, the corrected induced neural stem cells (ciNSCs) may be induced neural stem cells (HD-ciNSCs) including a mutated huntingtin (HTT) gene including an abnormal CAG repeat sequence, wherein the nucleotide sequence of SEQ ID NO: 2 included in the 5′ untranslated region (UTR) and exon 1 of the ciNSCs has been corrected.

1. A composition for correcting a huntingtin (HTT) gene, comprising a guide RNA comprising a nucleotide sequence complementary to the 5′ untranslated region (UTR) and first exon of the huntingtin (HTT) gene, or DNA encoding the guide RNA; and
a Cas protein or a polynucleotide encoding the same.

2. The composition of claim 1, wherein the 5′ untranslated region (UTR) and first exon of the huntingtin (HTT) gene comprise a nucleotide sequence of SEQ ID NO: 1.

3. The composition of claim 1, wherein the target sequence of the guide RNA comprises a sequence of 5-Nx-NGG-3 or 5-PAM(CCN)-Nx-3 included in the nucleotide sequence of SEQ ID NO: 1, wherein N is any one of A, G, C, and T, N in Nx is any one of A, G, C, and T, and x is a number within 20.

4. The composition of claim 1, wherein the target sequence of the guide RNA comprises a nucleotide of SEQ ID NO: 2.

5. The composition of claim 1, wherein the target sequence of the guide RNA is a mutated huntingtin (HTT) gene comprising an abnormal CAG repeat sequence.

6. The composition of claim 1, wherein the Cas protein is Cas9.

7. A method for correcting a huntingtin gene, the method comprising transducing DNA encoding a guide RNA including a nucleotide sequence complementary to the 5′ untranslated region (UTR) and first exon of the huntingtin (HTT) gene; and a polynucleotide encoding a Cas protein into induced neural stem cells (HD-ciNSCs) comprising a mutated huntingtin gene comprising an abnormal CAG repeat sequence.

8. The method of claim 7, wherein the transduction is performed using any one method of microinjection, electroporation, DEAE-dextran treatment, lipofection, nanoparticle-mediated transfection, protein transduction domain-mediated introduction, a viral or non-viral mediated gene delivery vehicle, and exosomes.

9. The method of claim 8, wherein the viral or non-viral mediated gene delivery vehicle is a viral vector or a plasmid.

10. The method of claim 7, wherein induced neural stem cells (HD-ciNSCs) comprising the mutated huntingtin (HTT) gene are induced to transdifferentiate from somatic cells of a Huntington's disease patient.

11. The method of claim 10, wherein the transdifferentiation comprises culturing somatic cells from a Huntington's disease patient in a medium comprising one or more of thiazovivin, valproic acid, purmorphamine, A8301, SB431542, deazaneplanocin A (DZNep), 5-AZA, and CHIR99021 in combination.

12. A pharmaceutical composition for preventing or treating Huntington's disease, comprising the induced neural stem cells (ciNSCs) corrected by the composition of claim 1.

13. The pharmaceutical composition of claim 12, wherein the corrected induced neural stem cells (ciNSCs) are induced neural stem cells (HD-ciNSCs) comprising a mutated huntingtin (HTT) gene comprising an abnormal CAG repeat sequence, wherein the nucleotide sequence of SEQ ID NO: 2 included in the 5′ untranslated region (UTR) and exon 1 of the ciNSCs has been corrected.

14. A method for correcting a huntingtin gene, the method comprising transducing DNA encoding a guide RNA including a nucleotide sequence complementary to the 5′ untranslated region (UTR) and first exon of the huntingtin (HTT) gene; and a polynucleotide encoding a Cas protein into induced neural stem cells (HD-ciNSCs) comprising a mutated huntingtin gene comprising an abnormal CAG repeat sequence to correct the huntingtin gene.

15. The method of claim 14, wherein induced neural stem cells (HD-ciNSCs) comprising the mutated huntingtin (HTT) gene are induced to transdifferentiate from somatic cells of a Huntington's disease patient.

16. The method of claim 15, wherein the transdifferentiation comprises culturing somatic cells from a Huntington's disease patient in a medium comprising one or more of thiazovivin, valproic acid, purmorphamine, A8301, SB431542, deazaneplanocin A (DZNep), 5-AZA, and CHIR99021 in combination.

17. The method of claim 14, wherein the corrected induced neural stem cells (ciNSCs) are induced neural stem cells (HID-ciNSCs) comprising a mutated huntingtin (HTT) gene comprising an abnormal CAG repeat sequence, wherein the nucleotide sequence of SEQ ID NO: 2 included in the 5′ untranslated region (UTR) and exon 1 of the ciNSCs has been corrected.
